(19) United States
(12) Patent Application Publication
(10) Pub. No.: US 2005/0196441 A1
(43) Pub. Date: Sep. 8, 2005

(54) Quick dissolving agrochemical and animal health products

(76) Inventors: James E. Dvorsky, Hilliard, OH (US); Brian Graham, Dublin, OH (US); Sreedhara Alavattam, Columbus, OH (US); Charles M. Gegenheimer, Columbus, OH (US); John R. Finney, Dublin, OH (US); Jean E. Schelhorn, Granville, OH (US); Dov S. Rosenberg, Bexley, OH (US); Jeffrey Cafmeyer, Columbus, OH (US); Mark R. Bauer, Delaware, OH (US); Robert J. Fellows, Richland, WA (US); Nadim C. Moucharafieh, El Sobrante, CA (US); Ronald Alan Coffee, Haslemere (GB); Alastair Bruce Pirrie, Oxford (GB); David Neville Davies, Oxford (GB); Johnathan Essex-Lopresti, Newbury (GB); Margaret Sin Ka Wan, Oxfordshire (GB); Anna Busby, Oxfordshire (GB)

Correspondence Address: DINSMORE & SHOHL LLP;One Dayton Centre, Suite 1300, One South Main Street, Dayton, OH, 45402-2023, US

(21) Appl. No.: 10/982,273

(22) Filed: Nov. 5, 2004

Related U.S. Application Data
(60) Provisional application No. 60/517,798, filed on Nov. 5, 2003.

(51) Int. Cl. A61K 9/46

(52) U.S. Cl. 424/466

(57) ABSTRACT

The present invention provides a manufacturing process for quick dissolving products having active ingredients directed to agrochemical applications, such as plant applications and animal and animal health applications. Electrohydrodynamic (EHD) spraying is used to form a product form preferably comprised of fibers that contains a desired agrochemical compound formulated to quickly disintegrate on contact with a solvent such as water. The dispersed chemical is then provided as a sprayable solution. Because the product form contains little or no liquid, significant size and weight savings are gained, higher active ingredient loadings may be placed in a smaller amount of material, and manufacturing, packaging and transportation costs are thereby reduced without impacting user-friendliness. In addition, the EHD manufacturing method of the present invention also permits combining active ingredients previously not capable of effective use together in a single product. Small, low cost and potentially modular production operations may be used to practice the invention, thus enabling production facilities to be located close to markets, and product design specific to local or regional markets.

This application claims priority to the following commonly assigned and co-pending: U.S. application Ser. No. 09/758,716, filed Jan. 11, 2001, having a PCT filing date of Jul. 22, 1997, entitled “Dispensing Device and Method for Forming Material”; U.S. application Ser. No. 10/018,160, filed Nov. 1, 2001, having a PCT filing date of May 5, 2000, entitled “Method and Apparatus for Manufacturing Dissolvable Tablets”; U.S. application Ser. No. 10/472,588, published as US 2004/0131673 on Jul. 8, 2004, and having a PCT filing date of Mar. 22, 2002, entitled “Manufacturing Dissolvable Dosage Forms”; PCT Application Ser. No. PCT/US04/00554, filed Jan. 10, 2004, published Jul. 29, 2004, (PCT Publication No. WO 04/062367) entitled “Sprayable Non-Aqueous, Oil-Continuous Microemulsions and Methods of Making Same;” U.S. application Ser. No. PCT/US03/33862, filed Oct. 27, 2003, entitled “Process For Treating Non-Human Animals;” and U.S. application Ser. No. 60/517,798, filed Nov. 5, 2003, entitled “Quick Dissolving Agrochemical And Animal Health Products”

### TECHNICAL FIELD

This invention relates in general to the manufacturing of quick dissolve agrochemical consumer and animal health products, and in particular to improved methods of forming products using electric field effect technology.

### BACKGROUND OF THE INVENTION

This invention relates generally to the field of agrochemical products, and more particularly to the use of electric field effect technology in the production of such products for use in providing active ingredients to plants and animals.

Many agrochemical products are supplied to the end user as a ready-to-use liquid formulation or a liquid concentrate that requires dilution with water. In both cases, the percentage of active ingredient is typically quite small compared to the overall volume of the product being supplied and purchased, the bulk of which is water. Hence, considerable weight in water is being packaged and shipped for purpose of providing an active ingredient in a user friendly form, with a resulting higher product, packaging and shipment cost to the consumer.

Some of the commercial and ready-to-use products currently available and many concentrates currently available are provided in a suitable solvent—generally water. These can be further mixed into water or applied directly by the consumer. Because many of the active ingredients in the agrochemical markets are highly active, the fraction of active ingredients, even in concentrated form, may amount to only a few percent of the total mass of the retail product. Hence, again, where concentrates are sold most of what is being processed, packaged, shipped, shelved, stored and sold is water.

Thus, while being consumer friendly, ready-to-use products are the least efficient in terms of space utilization and weight. Yet, concentrated products are often less favored by consumers because of the need for diluting and mixing and potential messiness, the requirement to work with and be exposed to dangerous substances, and the inaccuracies of measurement or dosing.

Alternative tablet products are available that are dissolved in water and applied by conventional spray technologies. However, such tablets are currently produced using either extrusion or compression. High temperatures associated with these processes can damage active ingredients, lessening the value of the end product.

Accordingly, there is a need for new products, methods and systems directed to improved agrochemical products which are consumer friendly, cost-effective and lower in cost.

### SUMMARY OF THE INVENTION

The present invention overcomes the disadvantages of previously known agrochemical products and tablet production processes by providing a unique manufacturing process for quick dissolving products having agrochemical active ingredients directed to plant applications and therapeutic agents directed to animal health applications. These two types of applications are generally referred to herein as “agrochemical applications”. The present invention permits production of quick dissolving products serving this broad range of agrochemical applications to be made in a user friendly form which isolates the active ingredients from the user.

In accordance with the present invention, electrohydrodynamic (EHD) spraying is used to form a solid or semi-solid or partially gel-like product, preferably substantially comprised of fibers, but which may also be comprised of fibrils, fiber fragments or segments, or particles, which contains a desired agrochemical active ingredient or therapeutic agent. EHD is also known as electric field effect technology (EFET), and is described generally in U.S. Pat. No. 6,252,129, issued Jun. 26, 2001, to Coffee, and elsewhere.

In accordance with the present invention, the product chemistry is formulated to quickly dissolve on contact with a solvent, preferably water, dispersing the active ingredient into a sprayable liquid product which, in turn, may be sprayed by a conventional spraying device, or an EHD spray device, such as those disclosed in co- pending an commonly assigned PCT Application Ser. No. PCT/US03/33862, filed Oct. 27, 2003, entitled “Process for Treating Non-Human Animals,” the disclosure of which is incorporated by reference herein in its entirety. The product structure is preferably generally porous, and preferably formed of fibers. The product form also can be structured to promote and maximize contact between the solvent and fibers. Because the fiber product contains little or no liquid, significant size and weight savings are gained. Higher active ingredient loadings may be placed in a smaller amount of material. As a result, manufacturing, packaging and transportation costs are thereby reduced without impacting user-friendliness.

The EHD manufacturing method of the present invention also permits the creation of fibers having different active ingredients, some of which are not capable of being effectively prepared for use together in a single product due to limited shelf life of one of the components when in a solvent, or due to chemical interactions that impact efficacy or cause settlement of suspended materials, or other interactions from the combination that limit shelf life. Thus, manufacturing methods in accordance with the present invention can enable use of materials in conventional sprayers that were not previously usable. Multiple layers can be produced and isolated or combined, and other manufacturing constraints apparent with conventional product manufacturing can be overcome, as detailed below.

Further, the product form of the present invention eliminates the need for measuring concentrated powders, granules or liquids, and further eliminates the danger of spillage and exposure during shipment as well as when the user is pouring product having active ingredients into a dispenser. The material can be produced in a dry or semi-dry, or gel-like pre-measured dosage units for dissolution in a solvent, adding to safety and convenience.

Finally, in accordance with the present invention, small, lower cost production operations may be used to practice the invention, thus enabling production facilities to be located close to markets, enabling product design specific to local or regional markets to be delivered to the marketplace. Moreover, modular production facilities may be developed in one location and shipped to multiple sites in a modular arrangement.

These and other objects and advantages of the present invention will be apparent in light of the description of the invention embodied herein.

### DETAILED DESCRIPTION OF THE PREFERRED EMBODIMENTS

The present invention contemplates systems and methods useful for quick dissolving format products (QDF products), also known as fast dispersion dosage forms (FDDFs). In the agrochemical applications of interest, QDF products preferably dissolve in less than one minute and some products dissolve in a matter of a few seconds.

The quick dissolve format product in accordance with the present invention, includes a polymer carrier which is soluble in a solvent, and an agrochemical active ingredient carried by the polymer carrier. At least one of the polymer carrier and agrochemical active ingredient are formed in the product by electrohydrodynamic processing. Where the QDF product is intended for use on plants the solvent for the agrochemical active ingredient is compatible with the plant application, and where the QDF product includes a therapeutic agent the solvent must be compatible with use on animals.

EHD (also known as EFET) is used to cone-jet spray a formulation containing an active ingredient, preferably as a fiber, onto a conveyor belt or other processing target. Preferably, the formulation is cone-jet sprayed with a polymer carrier or other carrier material. The sprayed material, again preferably a fiber, builds up to a mat that may be subsequently cut into unit dosage forms or sprayed into preformed molds, around shapes, or into other configurations. In this regard, co-pending and commonly assigned U.S. patent applications: Ser. No. 10/018,160, filed Nov. 1, 2001, entitled “Method and Apparatus for Manufacturing Dissolvable Tablets”: and Ser. No. 10/472,588, filed Nov. 18, 2002, entitled “Manufacturing Dissolvable Dosage Forms” (collectively referred to as the “QDP Applications”), detail processes to form the product in various forms including by way of example, tablets, strips and mats, the disclosures of which are incorporated by reference herein in their entirety. As well, EHD spraying devices and methods disclosed in U.S. Pat. No. 6,252,129, issued Jun. 26, 2001, to Coffee, the disclosure of which is incorporated herein by reference, are useful in the practice of the present invention and may be employed as taught in the QDP Applications.

In contrast with known QDF products for pharmaceuticals that use small oral dosage forms and have access to limited water in the oral cavity or other body cavities for dissolution, the present invention has the advantage of using larger amounts of solvents and a choice of both small and large QDF product sizes, thus taking advantage of some aspects of QDF materials, but providing additional advantages in agrochemical applications to plants and animals.

Further the size, weight and loading of active ingredient in a product made in accordance with the present invention is in sharp contrast with the size, weight and active ingredient loadings available in other forms of agrochemical products in the marketplace. Concentrates and tablets are known that have approximately a 15-20% active ingredient loading. Granules are known to exist in a range of approximately 10-35% active ingredient loading, and powders and dusts are below 35%. The EFET sprayed products of the present invention have had loadings of agrochemical active ingredients from 23% to over 82% by weight. Stated differently, the in accordance with the present invention, QDF products were produced having a ratio by weight of agrochemical active ingredient to the polymer carrier of from 0.3 to 1 up to greater than 4.5 to 1.

Thus, the methods of the present invention enable creation of a QDF product having much higher active ingredient loadings so that for a given volume of solvent, it may be possible to develop a higher concentration liquid product and spray. Higher concentration liquid products may advantageously be used in EHD dispensers which can create highly targeted and highly concentrated sprays, as taught in co-pending and commonly assigned PCT Application Ser. No. PCT/US03/33862, filed Oct. 27, 2003, entitled “Process for Treating Non-Human Animals.”

These alternative methods of producing and delivering an active ingredient formulation in accordance with the present invention offer the opportunity to provide lower cost alternatives, safer forms of delivery, controlled dosages, higher active ingredient loadings, and new combinations of active ingredients in existing agrochemical packaging for use in existing equipment, all benefiting the supplier, retailer and consumer. A QDF product will typically be small, lightweight and readily soluble in water or another solvent. The fast dissolution feature of the product assures the consumer a consistent and homogeneous solution without the need for extensive mixing or other processing.

QDF products can be packaged in containers of various kinds, flexible, rigid, or semi-rigid, sealed or unsealed, or with other features such as recloseability of the type found in pharmaceutical packaging. As well, the QDF product, once formed, can be coated with fibers, particles, dissolvable sealants or other like materials to enable the user to handle the product directly without exposure to active ingredients.

Upon opening a package containing the QDF product, in one embodiment of the invention, a consumer can drop the QDF tablet into a prescribed quantity of preferred solvent, such as water, and begin spraying the formulation with a conventional garden, plant or animal sprayer shortly thereafter. As described above, various packaging schemes can minimize handling of the agrochemical active ingredient, and the proper concentration of sprayable formulation is obtained when the prescribed volume of water or solvent is used. In another embodiment of the invention, the QDF product is inside a container, either loose, adhered to a surface by soluble adhesive, or sprayed directly onto interior surfaces, which is simply filled with a solvent such as water, assuring that the proper concentration is achieved. In this embodiment the container itself serves to prevent contact with the user and protects the product from degradation by the external environment until use. In a still further embodiment the container may also have a first volume adapted to receive the quick dissolve product before the step of mixing, and a second volume substantially isolated from the first volume and adapted to receive the solvent before the step of mixing. To achieve mixing, the user causes communication between the first and second chambers, thereby exposing the quick dissolve product to the solvent. The first and second volumes may be separated by a valve, frangible seal, or other means for defining separate volumes which may be removed, disabled or broken to permit mixing to occur. In still another embodiment of the invention, the QDF product is inside a container through which a solvent flows and wears away a portion of the QDF product substantially at a rate commensurate with the flow.

In a further embodiment intended to isolate the user from contact with the agrochemical active ingredient, the QDF product may be formed on one end of a stick, either on external surfaces or on inner surfaces of a hollow tube. In this embodiment, the stick or tube may be gripped at one end by the user to stir the QDF product of the present invention into a solvent in preparation for application. If the QDF product is on the exterior of the stick, it may be wrapped in a dissolvable polymer, and if on the interior of a tube, may be released for mixing by dissolvable portions on the tube, or dissolution of at least a portion of the lower end of the tube. In the tube configuration, milled QDF product in the form of a powder may be provided.

In addition to water, oil-based and organic solvents can be used. However, the vast majority of agrochemicals (80 to 90%) are insoluble in water. Thus, in many cases, the QDF product of the present invention will have a water-soluble polymer carrier, and the agrochemical active ingredient remains present in the liquid product as a suspension, emulsion or dispersion.

It has been found that some agrochemical active ingredients may advantageously be sprayed by using as a solvent a water continuous micro-emulsion of a non-aqueous solvent, as disclosed in commonly owned and co-pending PCT Application No PCT Application Ser. No. US04/000554, filed Jan. 10, 2004, “Sprayable Non-Aqueous, Oil-Continuous Microemulsions and Methods of Making Same,” the disclosure of which is incorporated herein by reference in its entirety. The non-aqueous phase may be used to enhance sprayability using an EHD sprayer, or alternatively, to substantially dissolve the active ingredient.

The QDF product of the present invention may be designed for dissolution by a wide range of solvents. By way of example and not limitation, typical solvents can include water, plant oils, dimethyl sulfonate, ethanol, methanol, isopropyl alcohol, acetone, hydrocarbons, and ethyl acetate.

In manufacturing a QDF product, the preferred fiber product of the present invention uses polyvinylpyrrolidone or another biocompatible polymer as the polymer carrier, which is formed into a fibrous matrix containing the active ingredient of interest.

Polymers that may be used for the carrier polymer include polymers of molecular weight from a few hundred to few hundred thousand. Suitable polymers are disclosed in U.S. Pat. No. 6,670,407, and may also be selected from synthetic biodegradable polymers disclosed in “Polymeric Biomaterials” ed. Severian Dumitriu, ISBN 0-8247- 8969-5, Publ. Marcel Dekker, New York, USA, 1994, synthetic non-biodegradable polymers, and natural polymers. Preferably the polymer is selected from homopolymers, block and random copolymers, polymeric blends and composites of monomers which may be straight chain, (hyper) branched or cross-linked.

As well many other polymers may be used such as: polyesters, including for example, poly(lactic acid), poly(glycolic acid), copolymers of lactic and glycolic acid, copolymers of lactic and glycolic acid with poly(ethylene glycol), poly(e-caprolactone), poly(3-hydroxybutyrate), poly(p-dioxanone), poly(propylene fumarate); Poly (ortho esters) including Polyol/diketene acetals addition polymers as described by Heller in: ACS Symposium Series 567, 292-305,1994; Polyanhydrides including poly(sebacic anhydride) (PSA), poly(carboxybisbarboxyphenoxyphenoxyhexane) (PCPP), poly[bis (p-carboxyphenoxy) methane] (PCPM), copolymers of SA, CPP and CPM, as described by Tamada and Langer in Journal of Biomaterials Science- Polymer Edition, 3, 315-353,1992 and by Domb in Chapter 8 of the Handbook of Biodegradable Polymers, ed. Domb A. J. and Wiseman R. M., Harwood Academic Publishers; Poly(amino acids); Poly(pseudo amino acids) including those described by James and Kohn in pages 389-403 of Controlled Drug Delivery Challenges and Strategies, American Chemical Society, Washington D.C.; Polyphosphazenes including derivatives of poly[(dichloro) phosphazene], poly[(organo) phosphazenes], polymers described by Schacht in Biotechnology and Bioengineering, 52,102-108,1996; and Azo polymers, Including those described by Lloyd in International Journal of Pharmaceutics, 106, 255-260, 1994.

Synthetic non-biodegradable polymers may be used, such as: Vinyl polymers including polyethylene, poly(ethylene-co-vinyl acetate), polypropylene, poly(vinyl chloride), poly(vinyl acetate), poly(vinyl alcohol) and copolymers of vinyl alcohol and vinyl acetate, poly(acrylic acid) poly(methacrylic acid), polyacrylamides, polymethacrylamides, polyacrylates, Poly(ethylene glycol), Poly(dimethyl siloxane), Polyurethanes, Polycarbonates, Polystyrene and derivatives. Other polymers which may be used in accordance with the present invention include: Polyacrylamides, which would help reduce drift and increase viscosity; Poly ethylene oxide; Poly vinyls; Poly ethylene glycol; Poly vinylpyrrolidone; Poly vinyl alcohol; Alkylated vinyl pyrrolidone polymers. The above mentioned polymers can be used alone or in combination with other polymers.

Natural Polymers may used, such as carbohydrates, polypeptides and proteins including: Starch, Cellulose and derivatives including ethylcellulose, methylcellulose, ethylhydroxyethylcellulose, sodium carboxymethylcellulose; Collagen; Gelatin; Dextran and derivatives; Alginates; Chitin; and Chitosan;

Preferably, a non biodegradable polymer, if used, is selected from polymers such as ester urethanes or epoxy, bis-maleimides, methacrylates such as methyl or glycidyl methacrylate, tri-methylene carbonate, di-methylene tri-methylene carbonate; and where used, biodegradable synthetic polymers such as glycolic acid, glycolide, lactic acid, lactide, p-dioxanone, dioxepanone, alkylene oxalates and caprolactones such as gamma-caprolactone are preferred.

The polymer may comprise any additional polymeric components having performance enhancing or controlling effect, for example determining the degree and nature of cross-linking for improved permeability by water or air, flexural and general mechanical properties.

The QDP Applications specifically teach production of quick dissolve pills for pharmaceutical applications using carrier polymers made from fish gelatin with a water-ethanol mix, food grade gelatins, polyvinyl pyridine, polyvinyl alcohol, poly-sucrose, other poly-saccharides such as starch and cellulose an its derivatives, sugars and confectionery mixtures such as toffee and caramel, and other biologically compatible products that can be formulated into a liquid solution or made liquid through the application of heat which will dissolve or melt on contact with wet surfaces. The QDP Applications also teach that mixtures of different polymers may also be used, for example a small quantity of another biologically acceptable polymer may be added to a gelatin formulation to improve its performance.

Other polymers mentioned in the QDP Applications include vinyl acetate, and vinylimidazole as present in Luviskol and Luvitec from BASF; PVP and its derivatives. However, as the QDP Applications focus on human and animal ingestion of pharmaceuticals, the broader list of polymers set forth herein include a wide range of additional polymers of interest to agrochemical applications.

As outlined in the QDP Applications, the active ingredient may be variously delivered into, onto or within the matrix, for example, suspended as a particle within the polymer carrier fibers, trapped among the fibers, adhered to the outside of the fibers, captured between layers of polymer fiber mats or solubilized with the fiber. When the active ingredient and polymer carrier are EHD processed as a single fluid, the agrochemical active ingredient tends to be imbedded in the fiber. When the active ingredient and polymer carrier are EHD processed contemporaneously through separate nozzles, coated particles and fibers can result, as well as fiber structures with active ingredients on the surfaces throughout the QDF product. Where the active ingredient and polymer carrier are EHD processed in series or alternating with each other, and alternating the charge applied to each component, layers will tend to form wherein one component tends to coat the other. Regardless, the structure and composition of the product is such that water can readily infuse through the interstices of the matrix and solubilize the polymer carrier and the active ingredient. This physical structure can be optimized somewhat to maximize dissolution effects on a given matrix and active material by choice of fiber diameter and fiber spacing. As well, an effervescing or similar dispersing agent may be added to the formulation to facilitate dissolution of the product in water. It has also been found that surfactants and viscosity enhancers may be added to assist in dissolution of the QDF product and distribution of the agrochemical active ingredient in the liquid product.

In accordance with the present invention, laboratory tests have shown that the active ingredient loading can be quite high—in excess of 80%, and it is expected that in some cases the active ingredient loading can approach 90% by weight, but more typically can be produced at a targeted loading of about 75%. This level of active ingredient loading is much higher than alternative conventional approaches employed in agrochemical applications, such as plant, animal or animal health applications, or in pharmaceutical processes for FDDFs.

Where therapeutic agents are provided in a QDF format, such agents may include medicaments for use in the treatment by way of therapy, surgery or diagnosis of humans or other animals. By way of example and not limitation, the therapeutic agents may include nicotine, morphine, a vitamin, an antiseptic, and anti-inflammatory, an antibiotic, an anti-cancer agent or other pharmaceutical product, veterinary medicines, vaccines, proteins, an enzyme, or genetic material such as DNA, cells and the like.

In accordance with one illustrative embodiment of the invention, QDF products are cut from a mat of material formed by EHD. In one configuration, the shape formed is 16 mm in diameter, 4 mm thick, weighs 270 mg and contains 210 mg of active material. In another configuration one gram of an insecticide to be dissolved in ten liters of water to yield the proper concentration of formulation to be sprayed, can be cut into a QDF product of any shape having a mass of roughly 1.3 grams and a volume of approximately four cubic centimeters (4 cc). Thus, for example, a product with this volume could be formed as a 2.25 cm diameter tablet that is 1 cm thick. As well, for improved dispersion, the tablet may have a hole in the middle, giving it a ring shape. If the outside diameter was increased to 2.5 cm (one inch), a 1 cm hole would still yield a 4 cc volume. Alternate configurations, such as dog-bone shapes, hollow balls, cylinders, spirals and the like may be desired for applications of interest involving plants or animals.

Compared to either 10 liters of a ready-to-use product (0.1% active ingredient) or 0.5 liters of 2% concentrate, the QDF product provides a far smaller package and weight, and yields significant cost reduction in packaging and handling.

Test Data for Exemplary QDF Product In accordance with the present invention, QDF products have been produced in the laboratory with an agrochemical active ingredient load of from 23% to 82%. The trial data below relates to a QDF product created wherein the agrochemical, Imidacloprid, was dispersed in a polymer solution of PVP, and EDH processed into fibers. This agrochemical active ingredient is illustrative of active ingredients that may be produced in accordance with the present invention, and is not intended to limit the invention thereto. The jet of liquid dried in flight to form a fiber that lays down on an earthed surface to form a low-density mat of fibers having a 3-D structure with large surface area. Additional surfactants were added at low concentrations to improve the dispersion of the active ingredient in the fibers thus created. Addition of any chemical that interacts with either the fiber formation or the polymer itself is intrusive to the EHD process and surfactants and dispersants were chosen that minimized impact on the EHD process, and that were approvable by the United States EPA. Thus, not all surfactants and dispersants will work. Target surfactants were chosen that at low concentrations had the ability to modify the surface tension of water to about 30 dynes/cm. Low concentrations of additives, including surfactants are required in order to keep the high loading (>50%) of the active ingredient in the PVP formulations.

Organosilicone surfactants, such as the Silwets, are a distinctive class of nonionic wetting agents which have the capacity to lower surface tension of aqueous mixes at concentrations (˜0.125%) lower than that of conventional organic nonionic surfactants (˜0.5 to 1.0%). These surfactants also spread more than conventional surfactants and provide improved effectiveness through maximum rainfastness. Pluronics are the other class of nonionic surfactants made of difunctional block copolymers and are relatively nontoxic. At concentrations of about 0.1%, Pluronics can reduce the surface tension of water to about 47 dynes/cm.

The Silwet and the Pluronics class of surfactants was sprayed via the EFET process using PVP as the fiber forming polymer to produce a QDF product. All the surfacants tested, Silwet-L7001, Silwet-L7200 and Pluronic L31 (average molecular weight 1100 Da) were conducive for EHD processing of the material.

While the use of surfactants would enhance rainfastedness and spreadability of the agrochemical on a leaf surface, they would probably not help if the chemical settles out fast in an aqueous media due to sedimentation. Dispersants such as polystyrene sulphonate and polysaccharides (gum arabic, xanthan gum, etc) were used to enhance the rate of dispersion in an aqueous media and to prevent rapid sedimentation of the active ingredient. This helped to put greater than 80% of the desired material solution for the agrochemical sedimentation assays. The ideal dispersant for use in the EFET process would be insoluble in EtOH, but rapidly soluble in water. However, dispersants that are soluble in both EtOH and water can be considered. Dispersants such as the BASF's Morwets (naphthalene sulfonic polycondensation products), Atlox Metaspheres etc can also be used for this purpose. All formulations were prepared in EtOH and sprayed using a single nozzle (point to plane) EFET device. A high voltage power supply and a syringe pump were used to provide the voltage required and to pump the liquid formulation through the nozzle respectively.

As well, viscosity enhancing reagents in the polymer solutions provided improvements in the quality of the QDF product. Some of the dispersants served also as viscosity enhancers. In particular, polysaccharide gums (gum arabicm, xantham gum, and the like), as well as polystyrene sulfonate, served well as viscosity enhancers in trials conducted in accordance with the present invention.

In addition, efferevescent agents were used to assist in dispersion, and a exemplary trial is included below where Citric Acid and Sodium Bicarbonate were included in the QDF product.

For testing the feasibility of these formulations, again, imidacloprid was the active ingredient and the following formulations (Table 1) were prepared and tested via EFET spraying. All samples sprayed well using EFET and formed non-friable fiber mats that had quick dissolve characteristics, dissolvable in ess than 60 seconds, when added to water.

The formulations below are illustrative of the range of loading achievable in accordance with the present invention, and other combinations are possible depending on the active ingredient, loading required in the final dose format and optimal dissolution time for the product.

TABLE 1
Formulations 1-10

| Ingredients (g) | 1 | 2 | 3 | 4 | 5 | 6 | 7 | 8 | 9 | 10 |
|---|---|---|---|---|---|---|---|---|---|---|
| PVP K90 | 7.52 | 7.5 | 10 | 10.01 | 1 | 1 | 0.5 | 0.5 | 4.5 | 1.5 |
| PVP K30 | 7.51 | 7.68 | 2.5 | 2.56 | 1 | 1 | 1 | 1 | 4.5 | 1.51 |
| Imidacloprid | 22.6 | 22.6 | 22.5 | 22.53 | 0.6 | 7 | 7 | 4 | 13.5 | 4.5 |
| Pluronic L31 | 2.5 | — | 2.05 | 2.03 | — | — | — | — | — | |
| Silwet L7001 | — | 2.5 | 0.5 | 0.55 | — | — | — | — | — | 0.5 |
| Poly styrene | — | — | 5 | — | — | — | — | — | — | |
| sulfonate | | | | | | | | | | |
| Gum Arabic | — | — | — | 5 | — | — | — | — | — | |
| Citric Acid | — | — | — | — | — | — | — | — | — | 0.25 |
| Sodium | — | — | — | — | — | — | — | — | | 0.25 |
| Bicarbonate | | | | | | | | | | |
| EtOH | 62.59 | 62.63 | 64.5 | 67.25 | 17.4 | 11 | 11.5 | 14.5 | 32.5 | 12.5 |
| Total (g) | 102.72 | 102.91 | 107.05 | 109.93 | 20 | 20 | 20 | 20 | 55 | 21.01 |
| % Imidacloprid | 56.32 | 56.11 | 52.88 | 52.79 | 23.08 | 77.78 | 82.35 | 72.73 | 60.00 | 52.9 |
| in fiber | | | | | | | | | | |

The fiber diameter and particle sizes of the QDF product produced included fibers having a diameter of about 3 to 5 microns, and particles of active ingredients of about 1 to 3 microns. These values are illustrative of QDF products which may have fiber diameters and particles sizes in the range from 1 micron and above, typically from 1 micron to 100 microns.

It was also observed that the same polymer of different molecular weight can be combined to effect dissolution rate In the solvent. Higher molecular weight PVP tends to be more flexible but slower in dissolution rate that the lower molecular weight PVP. Thus, the QDF product may be designed for a dissolution rate appropriate for the applications. Dissolution rates observed in the trials above ranged from approximately 30 seconds to 3 minutes.

Although not shown in the above trials, similar control and design of the dissolution rate in a solvent may be obtained by using two or more different polymers that will achieve both the desired structural properties and dissolution rate.

Time delayed dissolution in the environment, once a QDF product is dispensed to a target surface can be accomplished by encapsulating the agrochemical active ingredient in polymers or blends of polymers having different rates of dissolution in the environment.

Water, because of its convenience, availability and safety, is probably the most common solvent employed with agrochemicals and animal health products. However, the present invention is not limited to water as a dispersant and solvent. In some applications, organic solvents, such as ethanol or ethyl acetate, are more effective carriers for desired active ingredients. In some cases, agrochemical active ingredients are efficacious only in the presence of a solvent of the active ingredient, and not in a dry or powder form.

As well, compared to water-based formulations, oil-based formulations, such as those containing soybean or other plant oil, show greater adherence to an animal or a waxy leaf surface and provide improved rain-fastness. Both oil and organic solvents can more readily support EFET spraying (rather than pressure spraying) of the agrochemical in its actual application to the target. Surfactants may also be used with formulations to enhance their use, and can be included in the QDF product. The benefits of treating plants and animals with products sprayed using EFET have been documented co-pending and commonly assigned PCT Application Ser. No. PCT/US03/33862, filed Oct. 27, 2003, entitled “Process for Treating Non-Human Animals”, and are not the subject of the present invention.

In accordance with the present invention, an EFET-produced QDF can also include custom formulations for particular farming needs. Hence, multiple active ingredients may be formulated into a single QDF product, eliminating the need for the user to purchase different products or make multiple applications. Not only may multiple agrochemical active ingredients be formulated into a single QDF product, a mix of agrochemical active ingredients and therapeutic agents may be provided.

In accordance with the present invention, a kit comprised of a plurality of QDF products can be provide where various ones of the products have different agrochemical active ingredients or therapeutic agents. The desired pesticides specific to needs of the user can be selected from the kit and applied by spraying, brushing or other means to the desired surface.

Many of the agrochemical active ingredients, formulations and dose sizes may be designed for and unique to a specific region, season and/or crops. In a further aspect of the present invention, small cost-effective EFET-based manufacturing sites in desired locations may be built in order to create QDF products designed to serve an area's needs.

The process configurations depicted in the QDP Applications include a conveyor belt upon which the fiber mat is formed and an array of spray sites producing the fibers. The energy requirement to produce the high voltage needed to form the fibers is likely to be on the order of a few watts, even for a process having a hundred spray sites or more. Most of the power into the processing system is for moving the conveyor, pumping constituents being sprayed, such as a polymer carrier, active ingredient, solvent, inert material or other formulations, and conditioning the ambient environment, including the removal of any volatile solvent as they evaporate from the EHD spraying process.

The QDF product manufacturing process could overcome traditional agrochemical/animal health tablet formulation and/or manufacturing constraints such as, eliminating the need for high temperatures in a tablet extrusion process (which can be a limiting factor in a tablet extrusion process). Moreover, the EHD spraying process makes it possible to incorporate multiple active ingredients, opening up a number of additional product formulation options by co-spraying, layering coating materials, or mixing active ingredients, surfactants, carriers, coatings and other materials.

Among the types of products that may be made useful by producing QDF products in accordance with the present invention are unstable agrochemicals. These may be coated with a polymer before being processed by EHD into a QDF product, preserving their stability until the QDF product is prepared for application by a user. Examples of unstable agrochemicals include: Sulfonylureas (SU) and antifungals. Sulfonylureass are a class of compounds, typically containing aromatic and heterocyclic moieties bridged via a sulfonamide group. There is currently no liquid formulation for SU herbicides because of potential hydrolysis and photolysis. Photodegradation included breaking of a sulfonylurea bridge, as in the case of acidic hydrolysis and contraction of the sulfonylurea bridge was the major pathway of alkaline hydrolysis. Anti fungals (e.g. Famoxadone) are unstable in water. Famoxadone is an oxazolidinedione fungicide acknowledged for effective preventive effects and broad fungicidal spectrum. It is believed that antifungals may be useful in a QDF product form, where processed by EHD into fibers in a non aqueous media such as alcohol, acetone etc using PVP as carrier/encapsulant.

In the EHD processes used in accordance with the present invention, it is preferred to use polyvinylpyrrolidone (PVP) as a polymer carrier along with active ingredients, due to its solvency in ethanol, ease of fiber formation when EFET spraying techniques are used, and ability to dissolve in water in the final product. PVP is also an FDA approved material that is safe for ingestion, and consumer handling. Some conventional, compressed agrochemical products have been formulated with PVP. As well, some field studies suggest that PVP or other materials may protect active ingredients from degradation resulting from exposure to ultraviolet radiation. See U.S. Pat. No. 5,665,369, the disclosure of which is incorporated herein by reference, and The BCPC Conference -Pests & Diseases 2000, “A Unique Formulation of Alphacypermethrin with Enhanced Residual Properties and Precision Field Performance”, D. Marris, M. G. Ford, J. R. Smith, P. K. Leonard, P. E. Resner. Thus, in accordance with the present invention, EHD-formed fiber products including PVP can provide extended shelf life, enhanced by suspension of the active ingredient within the PVP fiber and potentially provide UV protection. Other improved product characteristics result from the manner in which an EHD spray deposits the active ingredient on the target surface, such as the availability or release profile of an active ingredient on a target surface.

While PVP is preferred, other polymer carriers may also be considered for this application. For rapid dispersion in water, the polymer carrier should be hydrophilic and compatible with the active ingredient. Other polymers, polymer carriers, or inert materials may be incorporated in the formulation to stabilize the solution, facilitate dispersion, improve shelf life, improve mechanical performance of spray droplet adhesion and deposition, and/or regulate the release of the active ingredient after it is applied to the target. A colorant may be employed to aid the user in distinguishing treated and untreated surfaces.

The present invention focuses upon a quick dispersing agrochemical product that is intended to rapidly disintegrate in water or other solvent for conventional pressure spraying or EFET-based spraying to plant or animals. However, it is also possible to formulate the dissolvable product into a slower releasing format that yields active ingredient based on the amount of solvent that flows over or through the product.

In accordance with this aspect of the present invention, the quick dissolving or quick dispersing form is placed directly into the sprayer where the solvent, most likely water, is intended to pass. As water flows through the sprayer, physical erosion and solubilization of the tablet contents occurs, forming a defined concentrate of active ingredient at the outlet of the sprayer. The product is replaced after it has fully disintegrated and become entrained and solubilized in fluid flow.

This aspect of the present invention is best suited for an application where the active ingredient is a fungicide, insecticide, herbicide, miticide or other form of pesticide or, as well, is a fertilizer, micro and macro nutrients, or other plant food.

In general a pesticide is a chemical or other substance produced and sold for the control of a pest species. A pesticide may kill the pest or merely inhibit its development. All substances sold to kill, retard, repel or attract pest species are regulated as pesticides. In the current version of the Federal Insecticide, Fungicide and Rodenticide Act, the legal definition of a pesticide has further been expanded to include defoliants, plant growth regulators, and desiccants.

Various types of pesticides are recognized by the types of pests they are used to control. Although most are chemical substances, some are either natural substances or synthetic versions of natural substances. As used herein, pesticide is intended to include the following categories:
Herbicide—a chemical or other substance used to kill undesirable plants.
Insecticide—a chemical or other substance used to kill undesirable insects.
Fungicide—a chemical or other substance used to kill undesirable fungi.
Miticide (also called acaricide)—a chemical or other substance used to kill mites and ticks.
Bactericide—a chemical or other substance used to kill bacteria (sometimes referred to as sanitizer or disinfectants).
Molluscicide—a chemical or other substance used to kill pest mollusks such as slugs and snails.
Nematicide—a chemical or other substance used to kill nematodes.
Plant growth regulator—a chemical or other substance used to desirably alter the growth processes of crop plants.
Wood preservative—a chemical substance used to protect wood from decay and stain fungi, insects, and other wood destroying organisms.
Defoliant—a chemical or other substance used to produce leaf drop.
Desiccant—a chemical or other substance used to promote drying as a harvest aid.

Among pesticides, there is a category of natural pesticides which suffer from shelf life stability issues when pre-mixed. In accordance with the present invention, these agrochemical active ingredients may be advantageously provided as a QDF product. By way of example and not limitation, such natural pesticides include Neem (Azadirachta indica). Neem is perceived as one of the environmentally safe alternative to synthetic pesticides. In spite of its strong biocidal properties against a wide range of crop pests, Neem products suffer from serious drawbacks of instability, slow action, short residual/shelf life, and quick degradation due to photolysis, thermolysis, hydrolysis and/or microbial/enzymatic degradation. Hence there is a need to improve efficacy and stability of bio or natural pesticides. In accordance with the present invention, such a pesticide can be coating with polymers such as PVP to increase their stability towards photolysis, hydrolysis and, it is believed, enzymatic degradation, enabling their use in a QDF product.

The agrochemical active ingredients or therapeutic agents in a QDF product are typically applied through spray means to the surface of a target, which may include plants, animals, soil, natural substrates or made-man structures and surfaces. The actual method of biochemical operation on those targets is not critical to the present invention and may be through contact or systemic means.

More generally, in accordance with the present invention, agrochemical active ingredients for plant applications or animal health applications further include, by way of example and not limitation, fertilizers, macro and micro nutrients.

Therapeutic agents include, by way of example and not limitation, veterinary biologics, health supplements and pharmaceuticals, including, but not limited to, animal vaccines, antibiotics, anti-inflammatories, chronic care medications, hormones, vitamins, birth control assistance drugs, and growth enhancers. These may be delivered to the target in a form produced in accordance with the present invention, with water added at or near the time of application, or as is preferred in accordance with the present invention, by dissolving the tablet and then spraying. In animal applications, transdermal delivery of active ingredients to animals is preferred. Cosmetic agents are those that modify the appearance or scent of an agrochemical target, i.e. a plant or animal, either visually, tactilely, or and include, by way of example and not limitation, cleaning agents, dyes, colorants, pigments, sheen enhancants, hair straightening compounds, hair detangling agents, deodorants, odorants and pheromones.

The present invention further includes methods of using the QDF product produced by EHD processing, which comprises the steps of providing an agrochemical active ingredient, and the step of mixing includes mixing the agrochemical active ingredient in at least a portion of the solvent. The method further includes placing the fluid product in contact with a dispensing device, and applying the fluid product to a target surface. As previously noted, spraying is preferred, but other methods such a brushing the liquid product onto a surface can also wok. It is possible to provide for timed release of an active ingredient in a QDF product, once it is dispensed. Polymers can be used to encapsulate agrochemical active ingredients to create a time release capability.

Although not preferred, in a further aspect of the invention, EHD processed carriers and agrochemical active ingredients may be milled to produce a powder or flowable powder, and the powder mixed with a solvent for application. In this alternative, it is preferred to provide EHD processed carriers with the agrochemical active ingredients embedded therein.

In another aspect of the invention, QDF products can be provided by layering techniques which permit a plurality of agrochemical active ingredients to chemically react once the QDF product is exposed to a solvent. This is particularly valuable in providing QDF products having agrochemical active ingredients that are not stable when together in a liquid product, or for those that react to produce a valuable pesticide where the reaction product has a short shelf life.

Further, the manufacturing platform of the present invention may be implemented in a modular fashion, with networkable systems, and the capability of splitting the manufacturing process across modules. Thus, while the processes disclosed in the QDP Applications could be disposed in a single transportable, turn-key unit, the scale of the spraying devices and subsequent conveyors could be such that multiple units could be produced and then integrated as a packaged, turn-key plant. This provides the opportunity for efficient manufacturing and deployment of multiple manufacturing sites.

Having described the invention in detail and by reference to preferred embodiments thereof, it will be apparent that modifications and variations are possible and logical changes may be made without departing from the spirit and scope of the invention.

1. A quick dissolve format product comprising:
a polymer carrier soluble in a solvent; and
a therapeutic agent carried by said polymer carrier;
wherein at least one of the polymer carrier and therapeutic agent are formed in the product by electrohydrodynamic processing.

2. The product of claim 1 wherein the therapeutic agent comprises one from the group consisting of: nicotine, morphine, a vitamin, an antiseptic, an anti-inflammatory, an antibiotic, an anti-cancer agent, pharmaceutical product, veterinary medicine, vaccine, protein, enzyme, genetic material, and combinations thereof.

3. The product of claim 1 further comprising an agrochemical active ingredient efficacious for human or animal use, wherein at least one of the polymer carrier, therapeutic agent and agrochemical active ingredient are formed in the product by electrohydrodynamic processing.

4. The product of claim 1 wherein the product includes a plurality of polymers.

5. The product of claim 4 wherein the plurality of polymers is the same polymer having different molecular weights.

6. The product of claim 4 wherein the product includes a plurality of polymers, at least one of the polymers generally encapsulating particles of the therapeutic agent, and at least one of the polymers serving as a polymer carrier.

7. The product of claim 6 wherein the polymers encapsulating particles of the therapeutic agent dissolve in the environment of use at various rates, thereby releasing the therapeutic agent over time.

8. A method of manufacturing tablets, comprising using electrohydrodynamic comminution to produce a dissolvable tablet.

9. A method of manufacturing tablets, comprising:
supplying to an outlet a liquid containing a biologically compatible carrier which melts or liquefies on contact with a solvent;
establishing an electric field between the outlet and a support surface to cause liquid issuing form the outlet to form at least one fiber or fibrils of the carrier;
causing the at least one fiber or fibrils to deposit onto the surface to form a fiber web or mat; and
providing the fiber web or mat with at least one therapeutic agent.

10. A kit comprised of a plurality of products formed according to the method of claim 1, wherein ones of the products have different therapeutic agents.

11. A method for using the product of claim 1 comprising:
providing the quick dissolve product of claim 1;
mixing the product with a portion of solvent, wherein the polymer carrier dissolves to produce a liquid product;
applying the liquid product to a target surface.

12. The method of claim 11 further comprising after the step of providing the product of claim 1, the step of milling the product to produce a powder.

13. A method of using the product of claim 11, wherein:
the step of providing is repeated to selectively provide two of said product; and
each selected product has a different therapeutic active ingredient.

14. A composition for a quick dissolve product comprising:
an EHD sprayable polymer having a molecular weight from 100 to 1,000,000; and
an EHD sprayable therapeutic agent.

15. The composition of claim 14 further comprising a surfactant.

16. The composition of claim 15 further comprising a dispersant.

17. The composition of claim 16 further comprising a viscosity enhancing agent.

18. The composition of claim 16 further comprising an effervescent agent.

19. The composition of claim 14 further comprising an EHD sprayable agrochemical active ingredient.
